(19) United States
(12) Patent Application Publication
(10) Pub. No.: US 2009/0324704 A1
(43) Pub. Date: Dec. 31, 2009

(54) PHOSPHATE DIESTER AMIDES

(75) Inventor: Hansjörg Eibl, Bovenden (DE)

Correspondence Address: ROTHWELL, FIGG, ERNST & MANBECK, P.C., 1425 K STREET, N.W., SUITE 800, WASHINGTON, DC, 20005, US

(73) Assignee: Max-Planck-Gesellschaft zur Foerderung Der Wissenschaften e.V., Muenchen (DE)

(21) Appl. No.: 12/306,356

(22) PCT Filed: Jun. 25, 2007
(86) PCT No.: PCT/EP07/05615

§ 371 Date: May 18, 2009

(30) Foreign Application Priority Data
Jun. 23, 2006 (DE) 10 2006 028 983.8

(51) Int. Cl. A61K 9/127; C07F 9/06

(52) U.S. Cl. 424/450; 558/166

(57) ABSTRACT

The present invention relates to lipid-analogous phosphoric acid compounds and in particular phosphoric triesters, phosphoric diester amides and phosphoric diesters. These are preferably cationic phospholipids preferably having at least one free hydroxyl group.

The present invention relates to lipid-analogous phosphoric acid compounds and in particular phosphoric triesters, phosphoric diester amides and phosphoric diesters. These are preferably cationic phospholipids preferably having at least one free hydroxyl group.

Phospholipids are highly interesting compounds which can be employed for a wide variety of purposes. Thus, for example, phospholipids are used as liposome constituents. However, they may also themselves represent active agents and be employed for the treatment of disorders or else for gene transfection. It was therefore an object of the present invention to provide further phospholipid structures which have favorable properties. This object is achieved according to the invention by a compound of the formula (I)

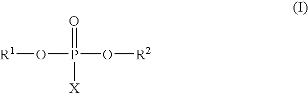

in which
R¹ is an apolar radical,
R² is —(CH₂)ₓ—N⁺(R⁴)₃, where
x=1 to 10, in particular 2 or 3,
R⁴ on each occurrence independently of one another H, alkyl which may optionally be substituted or may comprise heteroatoms, or has a meaning indicated for R², and
X is selected from —O—R³, —NR⁵—R³ and O⁻,
   where
R³ is an alkyl radical which may optionally be substituted and/or may include heteroatoms, or has a meaning indicated for R², and
R⁵ is in each case independently hydrogen, alkyl, OH or alkyl which is substituted or may comprise heteroatoms.

The compounds of the invention are lipid-analogous phosphoric acid compounds and in particular compounds having an overall positive charge. The compounds preferably have at least one positive excess charge. The positive charge may in this case be obtained by overcompensation of the negative charge or by elimination of the negative charge. An overcompensation of the negative charge can be obtained for example by introducing quaternary nitrogen atoms having a positive charge into the molecule. Elimination of the negative charge can take place for example by ester or amide formation.

The compounds of the invention further comprise preferably at least one free hydroxyl group per molecule, in particular at least two free hydroxyl groups per molecule, more preferably at least three free hydroxyl groups per molecule and even more preferably at least four free hydroxyl groups per molecule. The free hydroxyl groups result in particular in long circulation times in the body (liposomes) and stable complexes for gene transfection.

The phosphoric acid compounds of the invention include an apolar radical R¹ which constitutes in particular an apolar lipid structure. This radical R¹, which forms the hydrophobic part of the molecule, may be chosen from apolar radicals known to a person skilled in the art. R¹ is preferably a diacylglycerol, dialkylglycerol or acylalkylglycerol residue, where the acyl and alkyl groups preferably have 10 to 30, more preferably 12 to 28 and in particular 14 to 24 C atoms respectively. Diacyl or/and alkyl chains may be saturated or be mono- or polyunsaturated.

It is preferred in one embodiment to use naturally occurring fatty acid residues. In another embodiment, it is preferred to use acyl or alkyl radicals whose unsaturation is present at non-naturally occurring positions. Particularly preferred acyl radicals are radicals of oleic acid, linoleic acid or linolenic acid, and particularly preferred alkyl radicals are octadecenyl, octadecandienyl and octadecantrienyl radicals.

A particularly important embodiment is when R¹ simply represents an alkyl group, in particular C₁-C₃₀ alkyl, preferably C₈-C₂₈ alkyl, more preferably C₁₂-C₂₆ alkyl, most preferably C₁₄-C₂₄ alkyl. The alkyl group may be straight-chain or branched and saturated or unsaturated, in particular have one or two double bonds. The alkyl group may be for example tetradecyl, hexadecyl, octadecyl or preferably oleyl, linoleyl, or linolenyl. In this case, the alkyl radical is directly esterified with phosphate, i.e. glycerol is dispensed with as bridge molecule. The result in this embodiment is a class of compounds which has a direct pharmaceutical effect and can be employed against cancer and parasitic diseases.

The R² radical is according to the invention the —(CH₂)—N⁺(R⁴)₃ group. This radical introduces at least one positive charge into the molecule. x is preferably an integer from 1 to 10, in particular from 1 to 6 and particularly preferably 2 or 3. R⁴ is on each occurrence independently of one another hydrogen or an alkyl radical, where the alkyl radical may optionally be substituted or may comprise heteroatoms. It is preferably a C₁-C₁₀, in particular a C₁-C₆, more preferably a C₁-C₄ alkyl radical, in particular methyl. The alkyl radical may comprise one or more heteroatoms, in particular selected from oxygen, nitrogen and sulfur, preferably oxygen. R⁴ may furthermore be substituted by a substituent, in particular by one or more OH groups. R⁴ preferably has at least two, more preferably at least three OH groups. Such compounds in which R⁴ has an OH group represent a novel class of compounds. R⁴ may furthermore have the meaning indicated for R², whereby radicals of the —(CH₂)ₓ—N⁺(R⁴)₂—(CH₂)ₓ—N⁺(R⁴)₃ type may result. These radicals comprise two or more positive charges and thus lead to an overcompensation of the negative charge of the phosphate group. Such groups can be obtained starting from ethylenediamine for x=2, starting from propylenediamine for x=3, starting from butylenediamine for x=4, etc.

In a preferred embodiment of the invention, X is —NR⁵—R³. The compounds in this case are phosphoric diester amides. R³ is preferably an alkyl radical which may comprise heteroatoms and may optionally be substituted. Examples of suitable R³ radicals are for example methyl, ethyl etc. In a further preferred embodiment, R³ has the meanings indicated for R⁴. R³ is preferably an alkyl radical which is substituted by at least one OH group, more preferably by at least two OH groups, where the alkyl radical may comprise in the chain heteroatoms, especially nitrogen or oxygen, preferably oxygen. The alkyl radical preferably comprises 1 to 10, in particular 2 to 6, C atoms. R³ is particularly preferably —(CR⁵₂)_{y}—[O—(CR⁵₂)_{z}]ₚ—(CR⁵₃), where R⁵ is in each case independently hydrogen, OH, alkyl, in particular C₁-C₁₀-alkyl, more preferably C₁-C₆-alkyl and particularly preferably C₁-C₄-alkyl, optionally substituted, in particular by OH, y is an integer between 1 and 10, in particular from 1 to 6 and particularly preferably 2 or 3, z is an integer from 0 to 6, in particular 2 or 3, and p is an integer from 0 to 10, in particular from 1 to 5.

In a further preferred embodiment, X is —O—R³. The compounds in this case are phosphoric triesters. Such compounds preferably have at least one R⁴ radical which comprises at least one free hydroxyl group.

In a further preferred embodiment, X is O⁻. Compounds preferred in this case have at least one free hydroxyl group.

The compounds of the invention can be used in particular as liposome constituents. They can be employed in particular for producing liposomes with a positive excess charge (rapid accumulation in the liver).

However, they can themselves be employed as active agent, in particular as pharmaceutical. Especially when R¹=alkyl having 14 to 24 C atoms, the substances are active agents which are employed as pharmaceuticals.

The use of the compounds of the invention as a pharmaceutical in the treatment of cancer and of parasitic diseases, e.g. of leishmaniosis, is particularly important.

The compounds of the invention can additionally be used to prepare oligodeoxynucleotide (ODN) complexes and plasmid (DNA) complexes which can in turn themselves be used for transfection. The compounds are particularly suitable for transferring nucleic acids capable of expression (genes) or nucleic acids not capable of expression (oligodeoxynucleotides, ribozymes) into body cells with a therapeutic intention.

It has been found in particular that gene transfection in the serum is possible with the compounds of the invention including free OH groups, whereas with conventional compounds of the prior art it is necessary to remove the serum in the interim.

The invention further relates to a process for preparing a compound according to formula (I), comprising the steps:
(i) provision of O═PCl₃,
(ii) reaction of O═PCl₃ with an apolar compound, in particular with a diacylglycerol, a dialkyl-glycerol or an acylalkylglycerol,
(iii) reaction with a compound OH—R², where free amino groups present in R² are provided with a protective group,
(iv) reaction with HX and
(v) where appropriate removal of the protective group(s),
   where R² and X have the meanings indicated in claim 2.

Suitable protective groups are for example the BOC protective group. Since the third chlorine atom is very slow to react after the O═PCl₃ has been reacted with two alcohol functions, it is possible by using an amino alcohol for virtually quantitative reaction with the amine to take place, because the amine reacts about 1000 to 10 000 times more quickly than the alcohol group. It is possible by using amines which in turn include a plurality of free hydroxyl groups (e.g. NH₂—CH₂—CH(OH)—CH₂(OH)) without difficulty to introduce any desired number of free hydroxyl groups into the compounds of the invention.

The invention is further explained by the following examples.

### EXAMPLE 1

### Overcompensation of the Negative Charge on the Phosphate by Two N Atoms

### Apolar Radical

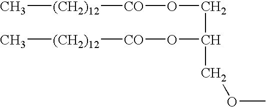

### Polar Modifications

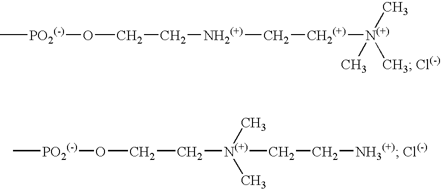

### Instead of Ethylenediamine

propylenediamine(diaminopropane)
butylenediamine(diaminobutane) etc.

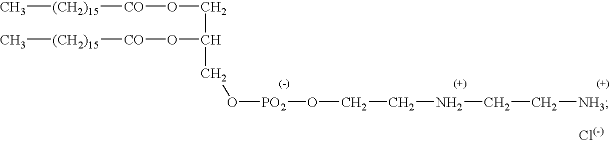

1,2-Dihexadecylglycero-3-phosphoethyl(ethylene-diammonium)

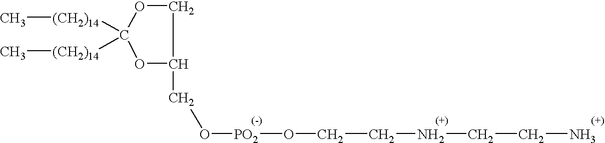

1,2-Dipentadecylmethylideneglycero-3-phospho(N-ethyl-amino)ethylammonium)

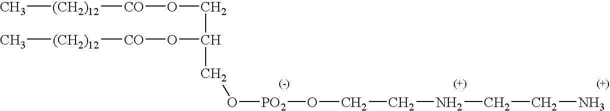

1,2-Dimyristoylglycero-3-phospho(N-ethylamino)(ethyl-ammonium)

### EXAMPLE 2

### Elimination of the Negative Charge on the Phosphate with Triester Formation

### Basic Structure

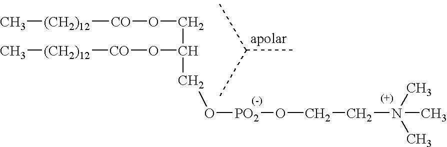

### Polar Modification (Ester)

two free hydroxyl groups

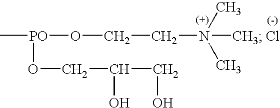

three free hydroxyl groups

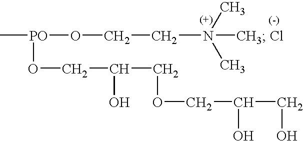

four free hydroxyl groups

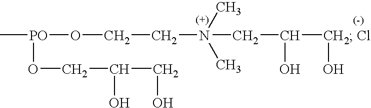

### EXAMPLE 3

### Elimination of the Negative Charge on the Phosphate by Diester Amide Formation

### Basic Structure

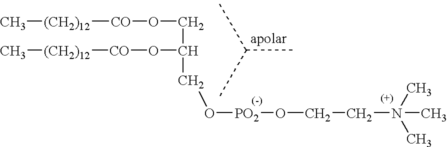

### Polar Modification (Amides)

one free hydroxyl group

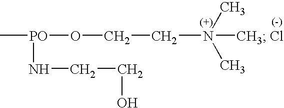

two free hydroxyl groups

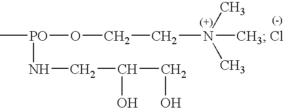

five free hydroxyl groups

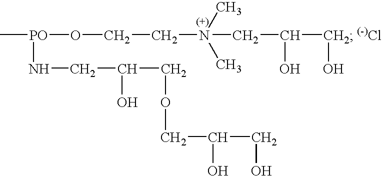

### EXAMPLE 4

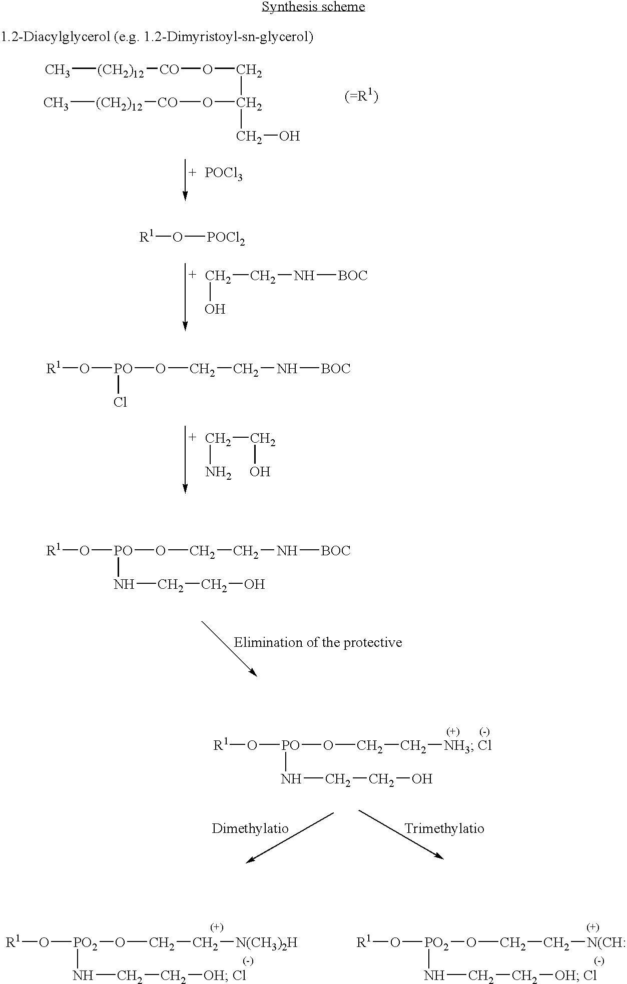

### EXAMPLE 5

### Preferred Compounds with Overcompensation of the Negative Charge for Use in Gene Transfection

1) 1,2-Dimyristoyl-sn-glycero-3-phospho-(N,N-di-methyl-N—[N′,N′,N′-trimethylethylammonio]ethyl-ammonium chloride,
   C₄₀H₈₂ClN₂O₈P (785.5)
   Detection by TLC comparison with the 1,2 dipalmitoyl derivative (see substance 2)
2) 1,2-Dipalmitoyl-sn-glycero-3-phospho-(N,N-dimethyl-N—[N′,N′,N′-trimethyl-ethylammonio]-ethylammonium chloride
   C₄₄H₉₀ClN₂O₈P (841.6)
   calc. (%): C, 62.79; H, 10.78; N, 3.33; P. 3.68
   found (%): 62.54; 10.71; 3.21; 3.67
3) 1,2-Distearoyl-sn-glycero-3-phospho-(N,N-dimethyl-N—[N′,N′,N′-trimethyl-ethylammonio]-ethylammonium chloride,
   C₄₈H₉₈ClN₂O₈P (897.8)
   Detection by TLC comparison with the 1.2 dipalmitoyl derivative (see substance 2)
4) 1,2-Dipalmitoyl-sn-glycero-3-phospho-(N,N-dimethyl-N—[N′,N′-dimethyl-ethylammonio]-ethyl-ammonium,
   C₄₃H₈₇N₂O₈P (791.2)
   calc. (%): C, 65.28; H, 11.09; N, 3.54; P. 3.92
   found (%): 65.11; 11.02; 3.47; 3.88
5) 1,2-Dioleyl-rac-glycero-3-phospho-(N,N-dimethyl-N—[N′,N′,N′-trimethyl-ethylammonio]-ethylammonium chloride,
   C₄₈H₉₈ClN₂O₆P (865.8)
   calc. (%): C, 66.59; H, 11.41; N, 3.24; P. 3.58
   found (%): 66.41; 11.35; 3.19; 3.45.

### EXAMPLE 6

Preferred novel compounds with overcompensation of the negative charge for use in gene transfection and for producing liposomes with positive excess charge.

For example, the result on use of 1,3-diamino-2-propanol is
6) 1,2-Dimyristoyl-sn-glycero-3-phospho-(N,N-di-methyl-N—[N′,N′-dimethyl-N′-hydroxypropylammonio]-ethylammonium chloride)
   C₄₁ H₈₄ Cl N₂ O₉ P (815.60)
      or with 1,4-diaminobutanediol-2.3
7) 1,2-Dimyristoyl-sn-glycero-3-phospho-(N,N-di-methyl-N—[N′,N′-dimethyl-N′-2,3-dihydroxypropyl-ammonio]-ethylammonium chloride)
   C₄₂H₈₅Cl N₂ O₁₀ P (845.80)

### EXAMPLE 7

### Positive Excess Charge Through Elimination of the Negative Charge on the Phosphate by Triester Formation

A) For Gene Transfection and for Producing Liposomes with a Positive Excess Charge
8) 1,2-Dimyristoyl-sn-glycero-3-phospho-ethylene glycol choline ester chloride
   C₃₈ H₇₇ Cl NO₉ P (758.46)
9) 1,2-Dimyristoyl-sn-glycero-3-phospho-glycerol choline ester chloride
   C₃₉H₇₉Cl NO₁₀ P (788.49)
10) 1,2-Dimyristoyl-sn-glycero-3-phospho-glycerol-(N,N,N-trimethyl)-propylammonium chloride
   C₄₉ H₈₁ Cl NO₁₀ P (802.51)
11) 1,2-Dipalmitoyl-sn-glycero-3-phospho-ethylene glycol choline ester chloride
   C₄₂ H₈₅ Cl NO₉ P (814.57)
12) 1,2-Dipalmitoyl-sn-glycero-3-phospho-glycerol choline ester chloride
   C₄₃H₈₇Cl NO₁₀ P (844.59)
13) 1,2-Dipalmitoyl-sn-glycero-3-phospho-glycerol-(N,N,N-trimethyl)-propylammonium chloride
   C₄₄H₈₉Cl NO₁₀ P (858.62)
14) 1-Palmitoyl-2-lauroyl-sn-glycero-3-phospho-ethylene glycol choline ester chloride
   C₃₈H₇₇Cl NO₉ P (758.46)
15) 1-Palmitoyl-2-lauroyl-sn-glycero-3-phospho-glycerol choline ester chloride
   C₃₉H₇₉ Cl NO₁₀ P (788.49)
16) 1-Palmitoyl-2-lauroyl-sn-glycero-3-phospho-glycerol-(N,N,N-trimethyl)-propylammonium chloride
   C₄₀ H₈₁ Cl NO₁₀ P (802.51)

### B) As Agent Against Cancer and Parasitic Diseases

17) Octadecyl-phospho-ethylene glycol choline ester chloride
   C₂₅ H₅₅ Cl NO₅ P (516.14)
18) Octadecyl-phospho-glycerol choline ester chloride
   C₂₆H₅₇ Cl NO₆ P (546.17)
19) Octadecyl-phospho-glycerol-(N,N,N-trimethyl)-propylammonium chloride
   C₂₇ H₅₉ Cl NO₆ P (560.20)
20) Oleyl-phospho-ethylene glycol choline ester chloride
   C₂₅ H₅₃ Cl NO₅ P (514.00)
21) Oleyl-phospho-glycerol choline ester chloride
   C₂₆H₅₅Cl NO₆ P (544.03)
22) Oleyl-phospho-glycerol-(N,N,N-trimethyl)-propyl-ammonium chloride
   C₂₇H₅₇Cl NO₆ P (558.06)
23) Erucyl-phospho-ethylene glycol choline ester chloride
   C₂₉ H₆₁ Cl NO₅ P (570.24)
24) Erucyl-phospho-glycerol choline ester chloride
   C₃₀ H₆₃ Cl NO₆ P (600.27)
25) Erucyl-phospho-glycerol-(N,N,N-trimethyl)-propyl-ammonium chloride
   C₃₁ H₆₅ Cl NO₆ P (614.30)

### EXAMPLE 8

Positive excess charge through elimination of the negative charge on the phosphate by amide formation.

A) For Gene Transfection and Production of Liposomes with Positive Excess Charge
26) 1,2-Dimyristoyl-sn-glycero-3-phospho-ethanolamide choline ester chloride
   C₃₈ H₇₈ Cl N₂ O₈ P (757.48)
27) 1,2-Dimyristoyl-sn-glycero-3-phospho-3-amido-1,2-propanediol choline ester chloride
   C₃₉ H₈₀ Cl N₂ O₉ P (787.46)
28) 1,2-Dimyristoyl-sn-glycero-3-phospho-3-amido-1,2-propanediol (N,N,N-trimethyl)propylammonium chloride
   C₄₀ H₈₂ Cl N₂ O₉ P (801.49)
29) 1,2-Dipalmitoyl-sn-glycero-3-phospho-ethanolamide choline ester chloride
   C₄₂ H₈₆ Cl N₂ O₈ P (813.55)
30) 1,2-Dipalmitoyl-sn-glycero-3-phospho-3-amido-1,2-propanediol choline ester chloride
   C₄₃ H₈₈ Cl N₂ O₉ P (843.57)
31) 1,2-Dipalmitoyl-sn-glycero-3-phospho-3-amido-1,2-propanediol (N,N,N-trimethyl)propylammonium chloride
   C₄₃ H₈₈ Cl N₂ O₉ P (857.60)
32) 1-Palmitoyl-2-lauroyl-sn-glycero-3-phospho-ethanolamide choline ester chloride
   C₃₈ H₇₈ Cl N₂ O₈ P (757.48)
33) 1-Palmitoyl-2-lauroyl-sn-glycero-3-phospho-3-amido-1,2-propanediol choline ester chloride
   C₃₉ H₈₀ Cl N₂ O₉ P (787.46)
34) 1-Palmitoyl-2-lauroyl-sn-glycero-3-phospho-3-amido-1,2-propanediol (N,N,N-trimethyl)propyl-ammonium chloride
   C₄₀ H₈₂ Cl N₂ O₉ P (801.49)

### A) As Agent Against Cancer and Parasitic Diseases

35) Octadecyl-phospho-ethanolamide choline ester chloride
   C₂₅ H₅₆ Cl N₂ O₄ P (515.12)
36) Octadeyl-phospho-3-amido-1,2-propanediol choline ester chloride
   C₂₆ H₅₈ Cl N₂ O₅ P (545.15)
37) Octadeyl-phospho-3-amido-1,2-propanediol (N,N,N-trimethyl)propylammonium chloride
   C₂₇ H₆₀ Cl N₂ O₅ P (559.18)
38) Oleyl-phospho-ethanolamide choline ester chloride
   C₂₅ H₅₄ Cl N₂ O₄ P (512.98)
39) Oleyl-phospho-3-amido-1,2-propanediol choline ester chloride
   C₂₆ H₅₆ Cl N₂ O₅ P (543.11)
40) Oleyl-phospho-3-amido-1,2-propanediol (N,N,N-tri-methyl)propylammonium chloride
   C₂₇ H₅₈ Cl N₂ O₅ P (557.04)
41) Erucyl-phospho-ethanolamide choline ester chloride
   C₂₉ H₆₂ Cl N₂ O₄ P (569.22)
42) Erucyl-phospho-3-amido-1,2-propanediol choline ester chloride
   C₃₀ H₆₄ Cl N₂ O₅ P (599.25)
43) Erucyl-phospho-3-amido-1,2-propanediol (N,N,N-tri-methyl)propylammonium chloride
   C₃₁ H₆₆ Cl N₂ O₅ P (613.28)

### EXAMPLE 9

Compounds having free hydroxyl groups on a radical including nitrogen; use for gene transfection and for producing liposomes with positive excess charge.

### A) As Triester

44) 1,2-Dimyristoyl-sn-glycero-3-phospho-glycerin-(N,N,-dimethyl-N-dihydroxypropyl)-ethylammonium chloride
   C₄₁ H₈₃ Cl NO₁₂ P (848.54)
45) 1,2-Dimyristoyl-sn-glycero-3-phospho-glycol-(N,N,-dimethyl-N-dihydroxypropyl)-ethylammonium chloride
   C₄₀ H₈₁ Cl NO₁₁ P (818.50)

### B) As Amide

46) 1,2-Dimyristoyl-sn-glycero-3-phospho-3-amido-1,2-propanediol-(N,N,-dimethyl-N-dihydroxypropyl)-ethylammonium chloride
   C₄₁ H₈₄ Cl N₂ O₁₁ P (847.52)
47) 1,2-Dimyristoyl-sn-glycero-3-phospho-glycol-(N,N,-dimethyl-N-dihydroxypropyl)-ethylammonium chloride
   C₄₀ H₈₂ Cl N₂ O₁₀ P (817.49)

1. A compound of the formula (I) 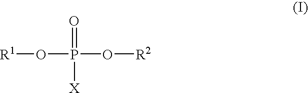
in which
is an apolar radical,
is —(CH₂)ₓ—N⁺(R⁴)₃, where
x=1 to 10, in particular 2 or 3,
R⁴ on each occurrence independently of one another H, alkyl which may optionally be substituted or may comprise heteroatoms, or has a meaning indicated for R², and
X is selected from —O—R³, —NR⁵—R³ and O⁻,
where
is an alkyl radical which may optionally be substituted and/or may include heteroatoms, or has a meaning indicated for R², and
R⁵ is in each case independently hydrogen, alkyl, OH or alkyl which is substituted or may comprise heteroatoms.

2. A compound of formula (I), characterized in that R³ is the radical
—(CR⁵₂)_{y}—[O—(CR⁵₂)_{z}]ₚ—(CR⁵₃)
where R⁵ is in each case independently of one another H, alkyl, OH or alkyl substituted by one or more OH groups,
y is an integer from 1 to 10, in particular from 1 to 6,
z is an integer from 0 to 10, in particular from 0 to 6, and
p is an integer from 0 to 10, in particular from 1 to 3.

3. A compound of the formula (I) as claimed in claim 1, characterized in that the compound overall has a positive charge.

4. A compound as claimed in claim 1, characterized in that it includes at least one, in particular at least two, preferably at least three, free hydroxyl groups.

5. A compound as claimed in claim 1, characterized in that R¹ is a diacylglycerol, a dialkylglycerol or an acylalkylglycerol residue.

6. A compound as claimed in claim 1, in which X is NR⁵—R³.

7. A compound as claimed in claim 1, characterized in that X is —O—R³.

8. A compound as claimed in claim 7, characterized in that at least one R⁴ radical comprises at least one OH group.

9. A compound as claimed in claim 1, in which X is O⁻.

10. A compound as claimed in claim 9, characterized in that it includes at least one free hydroxyl group.

11. A compound as claimed in claim 9, characterized in that at least one R⁴ radical has a meaning indicated for R².

12. A liposome comprising a compound as claimed in claim 1.

13. A pharmaceutical composition comprising a compound as claimed in claim 1.

14. The use of a compound as claimed in claim 1 for the manufacture of a pharmaceutical for the treatment of cancer.

15. The use of a compound as claimed in claim 1 for gene transfection.

16. A process for preparing a compound as claimed in claim 1, comprising the steps
(i) provision of O═PCl₃,
(ii) reaction of O═PCl₃ with an apolar compound, in particular with a diacylglycerol, a dialkylglycerol or an acylalkylglycerol,
(iii) reaction with a compound HO—R², where free amino groups present in R² are provided with a protective group,
(iv) reaction with HX and
(v) where appropriate removal of the protective group(s), where R² and X have the meanings indicated in claim 1.

17. A pharmaceutical composition comprising a liposome as claimed in claim 12.

18. The use of a liposome as claimed in claim 12 for the manufacture of a pharmaceutical for the treatment of cancer.

19. The use of a liposome as claimed in claim 12 for gene transfection.
